(19) United States
(12) Patent Application Publication
(10) Pub. No.: US 2005/0203102 A1
(43) Pub. Date: Sep. 15, 2005

(54) antibacterial agents

(75) Inventors: Shiv Kumar Agarwal, Chennai (IN); Surendrakumar Satyanarayan Pandey, Chennai (IN); Gajendra Singh, Chennai (IN); Santhanagopalan Chithra, Chennai (IN); Matte Marianna Samuel, Chennai (IN)

Correspondence Address: OLIFF & BERRIDGE, PLC, P.O. BOX 19928, ALEXANDRIA, VA, 22320, US

(73) Assignee: ORCHID CHEMICALS & PHARMACEUTICALS LTD., Chennai (IN)

(21) Appl. No.: 11/074,637

(22) Filed: Mar. 9, 2005

Related U.S. Application Data
(60) Provisional application No. 60/551,048, filed on Mar. 9, 2004.

(51) Int. Cl. A61K 31/496; C07D 417/14

(52) U.S. Cl. 514/254.02; 544/369; 514/151

(57) ABSTRACT

The present invention provides novel compounds of the general formula (I) and their pharmaceutically acceptable salts. The present invention more particularly provides novel oxazolidinone derivatives of the general formula (I).

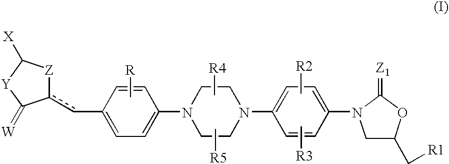

### FIELD OF THE INVENTION

The present invention provides novel compounds of the general formula (I) and their pharmaceutically acceptable salts. The present invention more particularly provides novel oxazolidinone derivatives of the general formula (I).

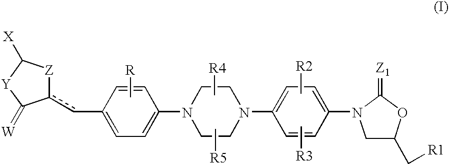

The novel oxazolidinone derivatives of the present invention may be useful as antibacterial agents and can be employed in the treatment of conditions such as Nosocomial Pneumoniae, Community Acquired Pneumoniae(CAP), and infection caused by Vancomycin Resistance Enterococci (VRE), Methicillin Resistance Staphylococcus Aureus (MRSA) and Penicillin Resistance Streptococcus Pneumoniae (PRSP).

### BACKGROUND OF INVENTION

Several oxazolidinone derivatives have been reported in the literature. Few prior art reference which disclose the closest oxazolidinone derivatives are given here:

U.S. Pat. No. 5,547,950 discloses and claims compounds of formula (IIa) 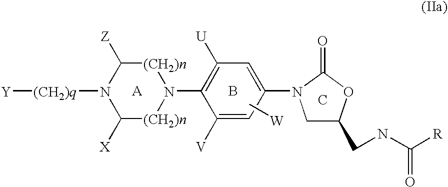
or pharmaceutically acceptable salts there of wherein each n is independently 1 to 3; Y is selected from a-n as defined in the patent; U, V and W are independently (C₁-C₆)alkyl, fluoro, chloro, bromo, hydrogen or a (C₁-C₆)alkyl substituted with one or more of fluoro, chloro, bromo or iodo, preferably U and V are fluoro and W is hydrogen; R is hydrogen, (C₁-C₁₂)alkyl, (C₃-C₁₂)cycloalkyl, (C₁-C₆)alkoxy, (C₁-C₆)alkyl substituted with one or more of fluoro, chloro, bromo, iodo or hydroxy and q is 0 to 4 inclusive.

WO 02/06278 describes a series of oxazolidinone derivatives useful as antimicrobial agents, of the formula (IIb) 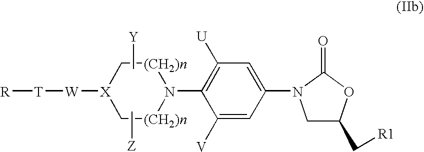
wherein T is a five to seven membered heterocyclic ring, aryl, substituted aryl; R is a substituent on T; X is CH₂, CH—S, CH—O and N; Y and Z are independently selected from hydrogen, alkyl, cycloalkyl; U and V are independently selected from alkyl, halogen; W is selected from group CH₂, CO, CH₂NH, CH₂NHCH₂, S, CH₂CO etc; R¹ is selected from −NH(C═O)R², wherein R² is hydrogen alkyl, cycloalkyl, alkoxy and the like.

U.S. publication No. 2002/0137754 describes a series of oxazolidinone derivatives useful as antimicrobial agents of the formula (IIc) 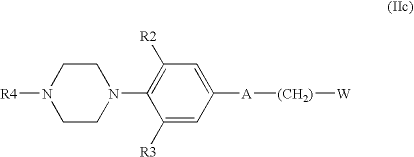
wherein A represents oxazolidinone ring and the like; W is NHC(═S)R¹, or —Y—het; Y is NH, O, or S; R¹ is H, NH_{2,} NHC₁-₄alkyl, C₁-₄alkenyl, etc; R² and R³ are independently H, F, Cl or C₁-₂alkyl; R⁴ is (a) —C(═O)—CR⁵R⁶—O—R⁷, (b) —C(═O)—CH₂S(O)n—CH₃, (c) —C(═O)—CH₂—S(═O)(═NR⁸)CH₃, (d) —C(═S)—R⁹, etc; R⁵ is H; R⁶ is phenyl, benzyl, etc, R⁷ is H, CH₃ or C₁-₄ alkanoyl; R⁸ is H, C₁-₄ alkyl, C₁-₄ alkanoyl, —C(═O)NH—C₁-₄ alkyl or —CO₂C₁-₄ alkyl; R⁹ is C₁-₄ alkyl, CH₂OR₁₁, S—C₁-₄ alkyl, OC₁-₄ alkyl, or NR¹²R¹³; R¹¹ is H, phenyl, benzyl, CH₃ etc; R¹² and R¹³ are independently H or C₁-₃ alkyl; or R¹² and R¹³ taken together form a 5- or 6- membered saturated heterocycle, wherein said saturated heterocycle may further contain one or two additional hetero-atoms selected from a group consisting of O, S(O)ₙ or NR⁷; n is 0, 1 or 2; and m is 0 or 1.

U.S. Pat. No. 6,342,513 and WO 00/32599 discloses compounds of the formula (IIc) 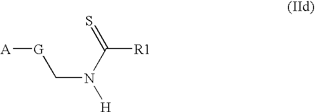
wherein G represents oxazolidinone ring and the like; R¹ is H, NH₂, NH alkyl, alkyl, alkoxy, etc, A is 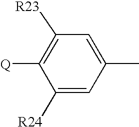
wherein R²³ and R²⁴ represents H, halogen and the like; Q is 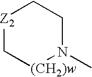
etc., wherein Z₂ is SO₂—, —O—, —(NR¹⁰⁷)—OS—, —S—, and the like; R¹⁰⁷ is —R¹⁰⁸CO—etc, R¹⁰⁸ is H, alkyl, aryl etc.

### OBJECTIVE OF THE INVENTION

We have focused our research to identify novel oxazolidinone derivatives, which are effective against resistant organisms. Our sustained efforts have resulted in novel oxazolidinone derivatives of the formula (I). The novel oxazolidinone derivatives of the present invention may be useful as antibacterial agents and can be employed in the treatment of conditions such as Nosocomial Pneumoniae, Community Acquired Pneumoniae(CAP), and infection caused by Vancomycin Resistance Enterococci (VRE) , Methicillin Resistance Staphylococcus Aureus (MRSA) and Penicillin Resistance Streptococcus Pneumoniae (PRSP).The compounds of the present invention are effective against a number of human or animal pathogens, clinical isolates, including Vancomycin resistant organisms, methicillin resistant organisms.

### SUMMARY OF THE INVENTION

The present invention relates to novel oxazolidinone derivatives of the formula (I) 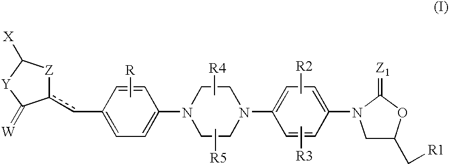
their pharmaceutically acceptable salts, wherein - - - - represents an optional bond; W represents O or S; Y represents NR^{9,} S or O, wherein R⁹ represents hydrogen, substituted or unsubstituted alkyl, alkenyl, —CH₂COOR¹⁰, or aryl, or counter ion; wherein R¹⁰ represents H or alkyl group; Z represents CR¹¹ or S; X represents ═O, ═S or together with R¹¹ forms fused 5 or 6 membered aromatic or heteroaromatic ring system containing carbon atoms or 1 or 2 heteroatoms selected from O, S or N; Z₁ represents O or S; R represents substituents selected from cyano, amino, alkyl, alkoxy, nitro, acyl, halogen atom, carboxylic acid or its esters; R¹ represents halogen, azido, nitro, cyano; AR^{6,} where A represents O or S, R⁶ represents hydrogen, substituted or unsubstituted groups selected from alkyl, cycloalkyl, aryl, aralkyl, acyl; N(R^{7a}R^{7b}) where R^{7a} and R^{7b} may be same or different and independently represent hydrogen, formyl, substituted or unsubstituted groups selected from (C₁-C₄)alkyl, aryl, aralkyl, heteroaryl, heteroaralkyl or an aminoacid residue which is attached through acid moiety, or R^{7a} and R^{7b} together with nitrogen may represent a mono or bicyclic saturated or unsaturated ring system which may contain one or more heteroatoms selected from O, S or N; or of the formula —NHC(═B)R⁸ wherein B represents O or S, R⁸ represents hydrogen, substituted or unsubstituted groups selected from (C₁-C₄)alkyl, (C₁-C₄)alkoxy, aryl, (C₃-C₆)cycloalkyl, amino, monoalkylamino, dialkylamino, arylamino, alkylcarbonylamino, arylcarbonylamino, heteroaryl, heterocyclyl, heteroaralkyl, or R¹ is of the formula —NIHS(O)ₚ(C₁-C₄)alkyl, —NHS(O)ₚaryl or —NHS(O)ₚheteroaryl, where p is 0 to 2; R² and R³ may be same or different and independently represent hydrogen, halogen, hydroxy, alkyl or alkoxy; R⁴ and R⁵ may be same or different and independently represent hydrogen, cyano, nitro, amino, halogen, hydroxy, substituted or unsubstituted groups selected from (C₁-C₄)alkyl, haloalkyl, (C₁-C₄)alkoxy, (C₁-C₄)alkylthio, (C₃-C₆)cycloalkyl or either of R⁴ or R⁵ represent an oxo or thiooxo group.

### DETAILED DESCRIPTION OF THE INVENTION

Suitable groups represented by R¹ may be selected from halogen atom such as fluorine, chlorine, bromine or iodine; azido, nitro, cyano, AR^{6,} N(R^{7a}R^{7b}), —NHC(═B)R⁸; —NHS(O)ₚ(C₁-C₄)alkyl, —NHS(O)ₚaryl or —NHS(O)ₚheteroaryl.

Suitable groups represented by X are selected from ═O, ═S; or together with R¹¹ forms fused 5 or 6 membered aromatic or heteroaromatic ring system containing carbon atoms or 1 or 2 hetereoatoms selected form O, S or N such as phenyl, naphthyl, furyl, pyrrolyl, pyridyl and the like.

Suitable groups represented by R² and R³ are selected from hydrogen, halogen atom such as fluorine, chlorine, bromine or iodine; hydroxy, (C₁-C₄)alkyl group such as methyl, ethyl, n-propyl, isopropyl, and the like; (C₁-C₄)alkoxy group, such as methoxy, ethoxy, n-propoxy, isopropoxy and the like.

Suitable groups represented by R⁴ and R⁵ are selected from hydrogen, cyano, nitro, amino, halogen, hydroxy, (C₁-C₄) alkyl group such as methyl, ethyl, n-propyl, isopropyl and the like oxo or thiooxo group.

Suitable groups represented by R⁶ are selected from hydrogen, substituted or unsubstituted linear or branched (C₁-C₄) alkyl group such as methyl, ethyl, n-propyl, isopropyl, n-butyl, isobutyl, t-butyl, and the like; (C₃-C₆) cycloalkyl group such as cyclopropyl, cyclobutyl, cyclopentyl, cyclohexyl and the like, which may be substituted; aryl group such as phenyl, naphthyl and the like, the aryl group may be substituted; aralkyl group such as phenylmethyl, phenylethyl, naphthylmethyl, naphthylethyl and the like, the aralkyl group may be substituted; acyl group such as —C(═O)CH₃, —C(═O)C₂H₅, —C(═O)C₃H₇, —C(═O)C₆H₁₃, benzoyl and the like, the acyl group may be substituted; thioacyl group such as —C(═S)CH₃, —C(═S)C₂H₅, —C(═S)C₃H₇, —C(═S)C₆H₁₃ and the like, the thioacyl group may be substituted; alkylsulfonyl group such as methylsulfonyl, ethylsulfonyl, and the like, which may be substituted; arylsulfonyl group such as phenylsulfonyl, naphthylsulfonyl and the like, which may be substituted; aralkylsulfonyl group such as phenylmethylsulfonyl, phenylethylsulfonyl, naphthylmethylsulfonyl, naphthylethylsulfonyl and the like, which may be substituted; heteroaryl group such as pyridyl, thienyl, furyl, pyrrolyl, oxazolyl, thiazolyl, imidazolyl, oxadiazolyl, triazolyl, tetrazolyl, pyrimidinyl, pyrazinyl, indolyl, indolinyl, benzothiazolyl, and the like, which may be substituted; heterocyclyl group such as pyrrolidinyl, thiazolidinyl, oxazolidinyl, morpholinyl, thiomorpholinyl, piperidinyl, piperazinyl, and the like, which may be substituted.

Suitable groups represented R^{7a} and R^{7b} are selected from hydrogen, formyl, substituted or unsubstituted linear or branched (C₁-C₄) alkyl group such as methyl, ethyl, n-propyl, isopropyl, n-butyl, and the like; aryl group such as phenyl, naphthyl and the like, which may be substituted; aralkyl group such as phenylmethyl, phenylethyl, and the like, which may be substituted; heteroaryl group such as pyridyl, thienyl, furyl, pyrrolyl, oxazolyl, thiazolyl, imidazolyl, isooxazolyl, oxadiazolyl, tetrazolyl, pyrimidinyl, pyrazinyl, pyridazinyl, benzopyranyl, indolyl, indolinyl and the like, which may be substituted; heteroaralkyl group wherein the heteroaryl moiety is as defined above; an aminoacid residue group selected from glycine, alanine, lysine, arginine, asparagine, aspartic acid, cysteine, glutamic acid, glutamine, histidine, iso-leucine, leucine, methionine, phenylalanine, proline, serine, threonine, tyyptophan, tyrosine or valine. Suitable ring systems formed by R^{7a} and R^{7b} together are selected from pyridyl, thienyl, pyrrolyl, oxazolyl, thiazolyl, imidazolyl, isooxazolyl, oxadiazolyl, triazolyl, thiadiazolyl, tetrazolyl, pyrimidinyl, pyrazinyl, pyridazinyl, benzimidazolyl, benzoxazolyl, benzothiazolyl, benzopyrrolyl, benzoxadiazolyl, benzothiadiazolyl, quinolinyl, dihydroquinolinyl, tetrahydroquinolinyl, isoquinolinyl, dihydroisoquinolinyl, tetrahydroisoquinolinyl and the like.

Suitable groups represented by R⁸ are selected from hydrogen, substituted or unsubstituted linear or branched (C₁-C₄) alkyl group such as methyl, ethyl, n-propyl, isopropyl, n-butyl, isobutyl, t-butyl and the like; (C₁ -C₄) alkoxy group, such as methoxy, ethoxy, n-propoxy, isopropoxy, butoxy and the like, which may be substituted; aryl group such as phenyl, naphthyl and the like, which may be substituted; (C₃-C₆)cycloalkyl group such as cyclopropyl, cyclobutyl, cyclopentyl, cyclohexyl and the like, which may be substituted; amino, which may be substituted; monoaikylanino group such as NHCH₃, NHC₂H₅, NHC₃H₇, NHC₆H₁₃, and the like, which may be substituted; dialkylamino group such as N(CH₃)₂, NCH₃(C₂H₅), N(C₂H₅)₂ and the like, which may be substituted; arylamino group such as phenylainio or naphthylamino, which may be substituted; alkylcarbonylamino group such as methylcarbonylamino, ethylcarbonylamino, n-propylcarbonylamino, iso-propylcarbonylamino and the like, which may be substituted; arylcarbonylamino group such as phenylcarbonylamino or naphthylcarbonylamino, which may be substituted; heteroaryl group such as pyridyl, thienyl, flryl, pyrrolyl, oxazolyl, thiazolyl, imidazolyl, isooxazolyl, oxadiazolyl, triazolyl, , pyrimidinyl, pyrazinyl, pyridazinyl, benzopyranyl, indolyl, indolinyl, and the like, which may be substituted; heterocyclyl group such as pyrrolidinyl, thiazolidinyl, oxazolidinyl, morpholinyl, thiomorpholinyl, piperidinyl, piperazinyl, and the like, which may be substituted; cycloalkyl amino group such as cyclopropyl amino, cyclobutylamino, cyclopentylamino, cyclohexylamino and the like, which may be substituted.

The substituents on any of the groups represented by R¹, R⁶, R^{7a}, R^{7b}, R⁸, are selected from halogen, hydroxy, formyl, nitro, cyano, azido, amino, alkyl, aryl, alkylamino, alkylaminocarbonyl, haloalkyl, acylamino, alkoxy, acyl and these substituents are as defined above. L represents a suitable leaving group selected from Fluoro, chloro, bromo, O—SO₂CH₃, O—SO₂Ph, O—SO₂C₆H₄—CH₃ and similar leaving groups.

Pharmaceutically acceptable salts of the present invention include alkali metal like Li, Na, and K, alkaline earth metal like Ca and Mg, salts of organic bases such as diethanolamine, a-phenylethylamine, benzylamine, piperidine, morpholine, pyridine, hydroxyethylpyrrolidine, hydroxyethylpiperidine, choline and the like, ammonium or substituted ammonium salts, aluminum salts. Salts also include amino acid salts such as glycine, alanine, cystine, cysteine, lysine, arginine, phenylalanine, guanidine etc. Salts may include acid addition salts where appropriate which are, sulphates, nitrates, phosphates, perchlorates, borates, hydrohalides, acetates, tartrates, maleates, citrates, succinates, palmoates, methanesulphonates, tosylates, benzoates, salicylates, hydroxynaphthoates, benzenesulfonates, ascorbates, glycerophosphates, ketoglutarates and the like. Pharmaceutically acceptable solvates may be hydrates or comprising other solvents of crystallization such as alcohols.

Representative compounds according to the present invention include:
(S)-N-[3-[3-fluoro-4-[4-[4-[(3-carboxymethyl-5-oxo-2-thioxo-1,3-thiazolidin-3-yl)methine]phenyl]piperazin-1 -yl]phenyl]-2-oxooxazolidin-5-ylmethyl] acetamide;
(S)-N-[3-[3-fluoro-4-[4-[4-[(1,3-dihydro-2-oxoindol-3-yl)methine]phenyl] piperazin-1-yl]phenyl]-2-oxooxazolidin-5-ylmethyl] acetamide;
(S)-N-[3-[3-fluoro-4-[4-[4-[(2-thioxo-4-oxo-1,3-thiazolidin-4-yl)methine] phenyl]piperazin-1 -yl]phenyl]-2-oxooxazolidin-5-ylmethyl]acetamide;
(S)-N-[3-[3-fluoro-4-[4-[4-[(2,4-dioxo-1,3-thiazolidin-4-yl)methine]phenyl] piperazin-1-yl]phenyl]-2-oxooxazolidin-5-ylmethyl] acetamide;
(S)-N-[3-[3-fluoro-4-[4-[4-[(1,3-dihydro-2-oxoindol-3-yl)methylene]phenyl] piperazin-l1-yl]phenyl]-2-oxooxazolidin-5-ylmethyl] acetamide;
(S)-N-[3-[3-fluoro-4-[4-[4-[(2-thioxo-4-oxo-1,3-thiazolidin-4-yl)methylene] phenyl]piperazin-1 -yl]phenyl]-2-oxooxazolidin-5-ylmethyl]acetamide;
(S)-N-[3-[3-fluoro-4-(4-(5-methylene-1,3-thiazolidine-2,4-dione)phenyl piperazin -1 -yl)phenyl]-1,3-oxazolidin-2-one-5- methylthiocarbamate;
(S)-N-[3-[3-fluoro-4-(4-(5-methylene-1,3-thiazolidine-2,4-dione )phenyl piperazin-1 -yl)phenyl]-1,3-oxazolidin-2-one-5- methylcarbamate;
(S)-N-[3-[3-fluoro-4-[4-[4-[(3-carboxymethyl-5-oxo-2-thioxo-1,3-thiazolidin-3-yl)methine]phenyl]piperazin-1 -yl]phenyl]-2-oxooxazolidin-5-ylmethyl] carbamate;
(S)-N-[3-[3-fluoro-4-[4-[4-[(5-oxo-2-thioxo-1,3-thiazolidin-3-yl)methine] phenyl]piperazin-1-yl]phenyl]-2-oxooxazolidin-5-ylmethyl] carbamate;
(S)-N-[3-[3-fluoro-4-[4-[4-[(3-carboxymethyl-5-oxo-2-thioxo-1,3-thiazolidin-3-yl)methine]phenyl]piperazin-1 -yl]phenyl]-2-oxooxazolidin-5-yl-ethyl] carbamate;
(S)-N-[3-[3-fluoro-4-[4-[4-[(5-oxo-2-thioxo-1,3-thiazolidin-3-yl) methine]phenyl]piperazin-1 -yl]phenyl]-2-oxooxazolidin-5-yl-ethyl] carbamate;
(S)-N-[3-[3-fluoro-4-[4-[4-[(1,3-dihydro-2-oxoindol-3-yl)methylene]phenyl] piperazin-1-yl]phenyl]-2-oxooxazolidin-5-ylmethyl] carbamate;
(S)-N-[3-[3-fluoro-4-[4-[4-[(1,3-dihydro-2-oxoindol-3-yl)methylene]phenyl] piperazin-1-yl]phenyl]-2-oxooxazolidin-5-yl-ethyl] carbamate;
(S)-N-[3-[3-fluoro-4-[4-[4-[(2-thioxo-4-oxo-1,3-thiazolidin-4-yl)methine] phenyl]piperazin-1-yl]phenyl]-2-oxooxazolidin-5-yl-ethyl] carbamate;
(S)-N-[3-[3-fluoro-4-[4-[4-[(2-thioxo-4-oxo-1,3-thiazolidin-4-yl)methine] phenyl]piperazin-1-yl]phenyl]-2-oxooxazolidin-5-ylmethyl] carbamate.

According to another embodiment of the present invention, there is provided a process for the preparation of novel oxazolidinone derivatives of the formula (I) wherein; and all other symbols are as defined earlier which comprises
i) Reacting the compound of the formula (IIIa) 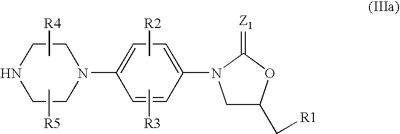
with compound of formula (IIIb) 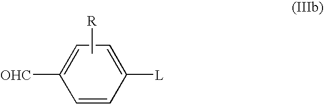
wherein L is a leaving group and R is as defined earlier to produce compound of formula (IIIc) 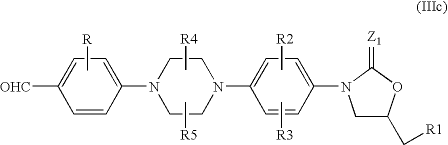
ii) reacting the compound of formula (IIIc) with compound of formula (IIId) 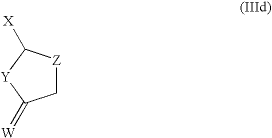
wherein all others groups are defined earlier to give the product compound of formula (I) and
iii) optionally reducing the compound of formula (I)

The reaction of compound of formula (IIIa) with compound of formula (IIIb) may be carried out in the presence of base such as sodium carbonate, potassium carbonate, triethyl amine, pyridine, DMAP, sodium hydroxide, potassium hydroxide and the like or mixture thereof and solvents such as toluene, dimethylformamide, tetrahydrofuran, chloroform, dichloromethane, dichloroethane, dioxane, ethylacetate, o-dichlorobenzene or a mixture thereof. The reaction may be carried out at a temperature in the range of 30° C. to 100° C. The duration of the reaction may range from 4 to 36 hr.

The reaction of compound of formula (IIIc) with compound of formula (IIId) may be carried out using benzoic acid and piperidine in the presence of solvents such as toluene, dimethylformamide, tetrahydrofuran, chloroform, dichloromethane, dichloroethane, benzoic acid, dioxane, ethylacetate, o-dichlorobenzene or a mixture thereof. The reaction may be carried out at a temperature in the range of 20° C. to reflux temperature. The duration of the reaction may range from 1 to 12 hrs.

The reduction of compound of formula (IIIg) may be carried out in the presence of gaseous hydrogen and a catalyst such as Ru, Pd, Rh, Pt, Ni on solid beads such as charcoal, alumina, asbestos and the like. The reduction may be conducted in the presence of a solvent such as dioxane, acetic acid, ethyl acetate, THF, alcohol such as methanol, ethanol, isopropanol and the like or mixtures thereof. A pressure between atmospheric pressure to 25 kg may be used. The reaction may be carried out at a temperature in the range of 25 to 100° C., preferably at room temperature. The reaction time ranges from 2 to 48 hr. The reduction may also be carried out by employing metal in mineral acids such as Sn/HCl, Fe/HCl, Zn/HCl, Zn/CH₃CO₂H and the like.

In yet another embodiment of the present invention, there is provided a process for the preparation of novel oxazolidinone derivatives of the formula (I) wherein R¹ represents —NHC(═B)R⁸ and all other symbols are as defined earlier, which comprises:
i) reacting a compound of the formula (IIIe) 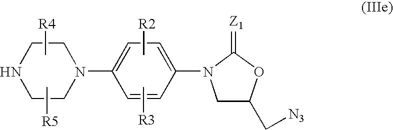
wherein all symbols are as defined earlier with a compound of formula (IIIB) 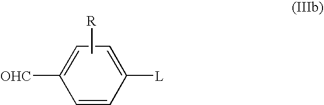
wherein L is a leaving group and all other symbols are as defined earlier to produce compound of formula (IIIf), 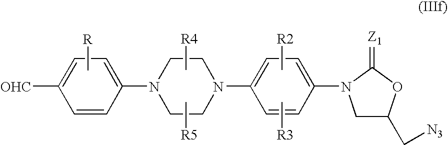
wherein all symbols are as defined earlier,
ii) reacting the compound of formula (IIIf) with compound of formula (IIId) 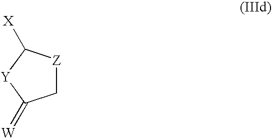
wherein X is as defined earlier to produce the compound of formula (IIIg) 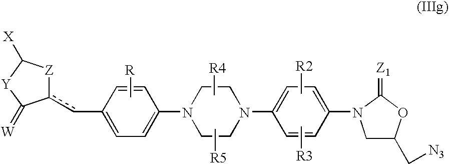
ii) reducing the compound of formula (IIIg) to a compound of formula (IIIh) 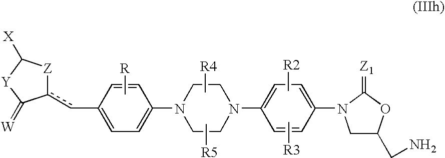
where all symbols are as defined earlier,
iii) acylating the compound of formula (IIIh) to produce compound of formula (I), where all symbols are as defined earlier.
iv) reducing the compound of formula (I) to compound of formula (I) .

The reaction of compound of formula (IIIe) with compound of formula (IIIb) may be carried out in the presence of base such as triethyl amine, pyridine, dimethyl amine, DMAP, and the like and solvents such as toluene, DMF, tetrahydrofuran, chloroform, dichloromethane, dichloroethane, ethylacetate, o-dichlorobenzene or a mixture thereof. The reaction may be carried out using reagent such as propyl-3-ethyl carbodimide hydrochloride, 1-ethyl-3-(3-dimethylaminopropyl)carbodiimide (EDAC), 1-hydroxybenztriazole hydrate (HOBt) and the like or mixture thereof. The reaction may be carried out at a temperature in the range of 20° C. to 50° C. The duration of the reaction may range from 1 to 12 hrs. The reaction of compound of formula (IIIf) with compound of formula (IIId) may be carried out using benzoic acid and piperidine in the presence of solvents such as toluene, DMF, tetrahydrofuran, chloroform, dichloromethane, dichloroethane, benzoic acid, dioxane, ethylacetate, o-dichlorobenzene or a mixture thereof. The reaction may be carried out at a temperature in the range of 20° C. to reflux temperature. The duration of the reaction may range from 1 to 12 hrs.

The reduction of compound of formula (IIIg) may be carried out in the presence of gaseous hydrogen and a catalyst such as Ru, Pd, Rh, Pt, Ni on solid beads such as charcoal, alumina, asbestos and the like. The reduction may be conducted in the presence of a solvent such as dioxane, acetic acid, ethyl acetate, THF, alcohol such as methanol, ethanol, isopropanol and the like or mixtures thereof. A pressure between atmospheric pressure to 25 kg may be used. The reaction may be carried out at a temperature in the range of 25 to 100° C., preferably at room temperature. The reaction time ranges from 2 to 48 hrs. The reduction may also be carried out by employing metal in mineral acids such as Sn/HCl, Fe/HCl, Zn/HCl, Zn/CH₃CO₂H and the like.

Acylation of compound of formula (IIIh) may be carried out using acylating agents such as anhydrides like acetic anhydride, propionic anhydride, acid chlorides like acetyl chloride, propionyl chloride, thioacids such as thioacetic acid or the reaction is careid out in the presence of alkyl chloroffromate such as methylchloroformate , ethylchloroformate and the like. The reaction may be carried out in the presence of appropriate solvents like tetrahydrofuran, chloroform, dichloromethane, dichloroethane, ethylacetate, o-dichlorobenzene or a mixture thereof. The reaction may be carried out in the presence of a base selected from DMAP, triethylamine, pyridine and the like. The reaction may be carried out at a temperature in the range of 0° C. to 50° C. The duration of the reaction may range from 6 to 24 hrs. It is appreciated that in any of the above-mentioned reactions, any reactive group in the substrate molecule may be protected according to conventional chemical practice. Suitable protecting groups in any of the above-mentioned reactions are those used conventionally in the art. The methods of formation and removal of such protecting groups are those conventional methods appropriate to the molecule being protected.

The present invention is provided by the examples below, which are provided by way of illustration only and should not be considered to limit the scope of the invention.

### EXAMPLE 1

### Preparation of (S)-N- [3-[3-fluoro-4-[4-{(4-formyl) phenyl }piperazin-1-yl] phenyll -2-oxo-oxazolidin-5-ylmethyll acetamide

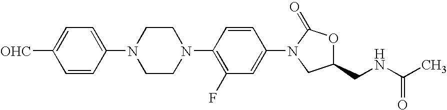

To a solution of (S)-N-[3-[3-fluoro-4-[piperazin-1-yl]phenyl]-2-oxo-oxazolidin-5-ylmethyl]acetamide (Prepared according to the procedure described in Journal of Medicinal Chemistry 1996, vol 39, No. 3, 673-679) (1.2 g, 3.57 mmol) in dried dimethylformamide (15 ml), potassium carbonate (2.9 g, 21 mmol) was added and stirred for 10 minutes under nitrogen atmosphere at 30° C. To this reaction mixture p-fluoro benzaldehyde (2.9 g, 8.9 mmol) was added slowly and stirred further at 70° C. for 24 hr while monitoring by TLC. The reaction mixture was quenched with cold water, extracted with ethyl acetate. The organic layer was dried over sodium sulfate and concentrated to afford the title compound. Yield: 0.55 g; ¹H-NMR (DMSO-d₆): δ 1.8 (s, 3H), 3.1 (m, 4H), 3.4 (t, 2H), 3.5 (m, 4H), 3.6 (m, 1H), 4.0 (t, 1H), 4.7 (m, 1H), 7.1 (m, 4H), 7.4 (dd, 1H), 7.7 (d, 2H). 8.2 (t, 1H), 9.7 (s, 1H); M/z^{m+1}: 441

### EXAMPLE 2

### Synthesis of (S)-N-[3-[3-fluoro-4-[4-[4-[(3-carboxymethyl-5-oxo-2-thioxo-1,3-thiazolidin-3-yl)methinelphenyllpiperazin-1-yllphenyll-2-oxo-oxazolidin-5-ylmethyll acetamide

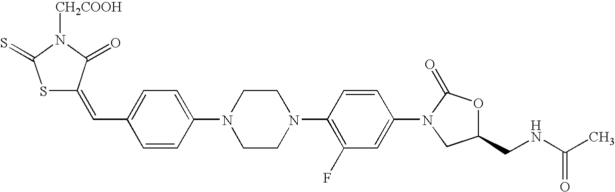

To a solution (S)-N-[3-[3-fluoro-4 -[4-{(4-formyl) phenyl}piperazin-1-yl]phenyl]-2-oxo-oxazolidin-5-ylmethyl]acetamide (100 mg, 0.2 mmol) in toluene, rhodanine-N-acetic acid (75 mg, 0.39 mmol), benzoic acid (2 mg, 0.38 mmol) and piperidine (2 mg, 0.028 mmol) were added and refluxed the reaction mixture for 1 hr. The solid thus separated on cooling to room temperature, was filtered and dried to furnish the title compound as red solid. Yield: 0.120 g; ¹H-NMR (DMSO-d₆): δ 1.8 (s, 3H), 2.9 (s, 4H), 3.1 (s, 4H), 3.5 (s, 5H), 3.7 (t, 1H), 4.1 (t, 1H), 4.2 (s, 2H), 4.3 (s, 1H), 7.1 (m, 4H), 7.5 (m, 3H), 7.7 (s, 1H), 8.4 (s, 1H); M/z^{m+1}: 613.

The following compounds were prepared according to the procedure given in example 2.

| Example No. | Structure | Analytical Data |
|---|---|---|
| 3 | 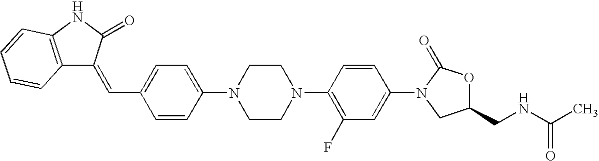 | Yield: 0.110 g; ¹H-NMR (DMSO-d₆): δ 1.8 (s, 3H), 3.1 (d, 4H), 3.5 (d, 4H), 3.7 (d, 1H), 4.0 (t, 1H), 4.7 (m, 1H), 6.7 (d, 1H), 6.8 (d, 1H), 6.9 (m, 5H), 7.5 (d, 1H), 7.6 (t, 3H), 8.2 (s, 1H), 8.4 (d, 1H), 10.5 (s, 1H); M/z^{m+1}: 556. |
| 4 | 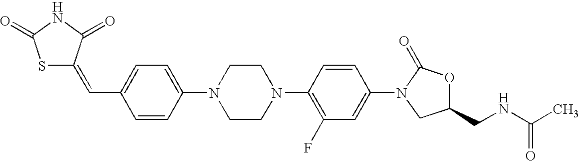 | Yield: 0.115 g; ¹H-NMR (DMSO-d₆): δ 1.8 (s, 3H), 2.9 (d, 4H), 3.3 (d, 5H), 3.6 (d, 2H), 4.0 (t, 1H), 4.7 (d, 1H), 7.1 (m, 4H), 7.4 (m, 3H), 8.3 (s, 1H), 9.6 (s, 1H); M/z^{m+1}: 540. |
| 5 | 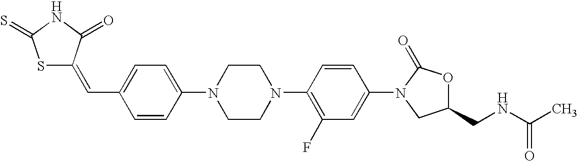 | Yield: 0.130 g; ¹H-NMR (DMSO-d₆): δ 1.8 (s, 3H), 3.1 (d, 4H), 3.4 (d, 5H), 3.5 (t, 1H), 3.7 (t, 1H), 4.0 (d, 1H), 4.7 (m, 1H), 7.1 (m, 4H), 7.4 (m, 4H), 8.2 (d, 1H); M/z^{m+1}: 556. |

### EXAMPLE 6

### Synthesis of (S)-N-[3-[3-fluoro-4-[4-[4-[(1,3-dihydro-2-oxoindol-3-yl)methylenelphenyllpiperazin-1-yl]phenyl]-2-oxooxazolidin-5-ylmethyll acetamide

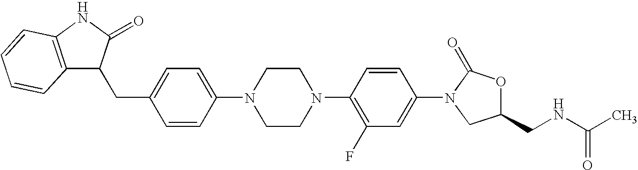

To a solution of (S)-N-[3-[3-fluoro-4-[4-{(3)-3-benzylidene-1,3-dihydro-2H-indol-2-one} piperazin-1 -yl]phenyl]-2-oxooxazolidin-5-ylmethyl] acetamide (500 mg, 0.9 mmol) in tetrahydrofuran (200 ml) and methanol (100 ml), 10% Pd/C (100 mg) was added and stirred under 10 kg pressure of hydrogen for 24 hr. The reaction mixture was filtered and concentrated to get crude product. The crude product was washed with ethyl acetate to furnish the title compound as off white solid. Yield: 0.400 g; ¹H-NMR (DMSO-d₆): δ 1.8 (s, 3H), 2.8 (q, 1H), 3.0 (d, 4H), 3.2 (d, 5H), 3.3 (m, 3H), 3.7 (m, 2H), 4.7 (t, 1H), 5.7 (s, 1H), 6.7 (d, 1H), 6.8 (m, 3H), 6.9 (d, 1H), 7.0 (t, 2H), 7.1 (t, 2H), 7.4 (s, 1H), 7.5 (s, 1H), 8.2 (t, 1H), 10.2 (s, 1H); M/z^{m+1}: 558.

The following compound was prepared according to the procedure given in example 6.

| Example No. | Structure | Analytical Data |
|---|---|---|
| 7 | 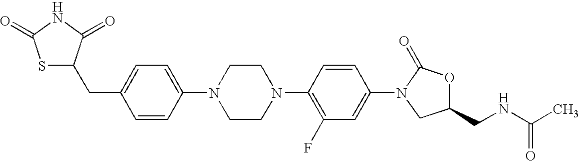 | Yield: 0.160 g; ¹H-NMR (DMSO-d₆): δ 1.8 (s, 3H), 3.1 (m, 4H), 3.27 (m, 5H), 3.4 (m, 3H), 3.5 (t, 1H), 3.7 (m, 2H), 4.0 (t, 1H), 4.71 (m, 1H), 6.94 (d, 2H), 7.1 (m, 3H), 7.20 (d, 1H), 7.53 (d, 1H), 8.2 (t, 1H); M/z^{m+1}: 542.3. |

### EXAMPLE 8

### Preparation of (S)-N-3-[3-fluoro-4 -(4-(5-methylene-1,3-thiazolidine-2,4-dione) phenylpiperazin-1-yl)phenyll-1,3-oxazolidin-2-one-5-methylthio carbamate

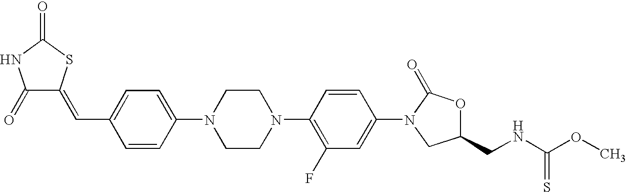

### STEP I

### Synthesis of (S)-N-5-(azidomethyl)-3-(3-fluoro-4-(1-benzaldehyde-4-yl)-piperazin-1-yl-phenyl)-1,3-oxazolidin-2-one

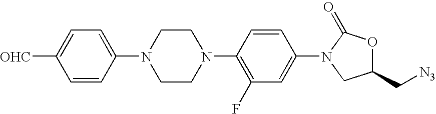

To a solution of (s)-N-5-(azidomethyl)-3-(3-fluoro-4-piperazin-1-ylphenyl)-1,3-oxazolidin-2-one (Journal of Medicinal Chemistry 1996, vol 39, No. 3, 673-679) (9.7 g; 30.3 mmol) in dry dimethylformamide (200 ml), potassium carbonate (41.9 g; 303.5 mmol) was added and stirred for 10 minutes under nitrogen atmosphere at 30° C. To this 4-fluoro benzaldehyde (11.27 g; 90.88 mmol) was added and stirred further at 75-80° C. for 40 hr while monitoring the TLC. After completion of reaction, the reaction mixture was quenched with water and extracted with ethyl acetate. Organic layer was dried over anhydrous sodium sulfate and concentrated to afford the title compound. Yield: 7.6 g; M/z^{m+1}: 425.2.

### STEP II

### Synthesis of (s)-N-15-(azidomethyl)-3-[3-fluoro-4-(4-(5-methylene-1,3-thiazolidine-2,4-dione )phenylpiperazin-1-yl)phenyll-1,3-oxazolidin-2-one

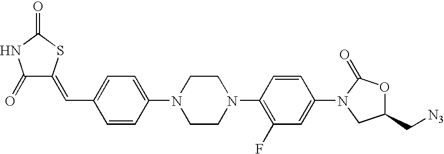

To a solution of (S)-N-5-(azidomethyl)-3-(3-fluoro-4-(1-benzaldehyde-4-yl)-piperazin-1-yl-phenyl)-1,3-oxazolidin-2-one (1.0 g; 2.35 mmol) in toluene(50 ml) , 2,4-thiazoldinedione (0.331 g, 2.83 mmol), benzoic acid (0.043 g, 0.35 mmol) and piperidine (0.027 g, 0.31 mmol) were added and refluxed the reaction mixture with continuous removal of water using dean stark while monitoring by TLC. The reaction mixture was allowed to cool to 30° C., the solid separated was filtered and dried to gave product. Yield: 1.17 g; M/z^{m+1}: 524.2.

### STEP III

### Synthesis of (S)-N-[5-(aminomethyl)-3-[3-fluoro-4-(4-(5-methylene-1,3-thiazolidine-2,4-dione )phenylpiperazin-1-yl)phenyll-1,3-oxazolidin-2-one

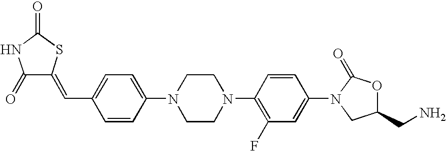

To a solution of (S)-N-[5-(azidomethyl)-3-[3-fluoro-4-(4-(5-methylene-1,3-thiazolidine-2,4-dione)phenylpiperazin-1 -yl)phenyl]-1,3-oxazolidin-2-one (0.410 g ; 0.78 mmol) in tetrahydrofuran (150 ml) was added 0.100 g of 10% Pd/C and the reaction mixture was hydrogenated at 30 psi for 3 hr . After completion of reaction, the reaction mixture was filtered and concentrated the organic layer to afford title compound. Yield: 0.385 g; M/z^{m+1}: 497.9.

### STEP IV

### Synthesis of (S)-N-5-(isothiocyanatomethyl)-3-[3-fluoro-4-(4-(5-methylene-1,3-thiazolidine-2,4-dione )phenylpiperazin-1-yl)phenyll-1,3-oxazolidin-2-one

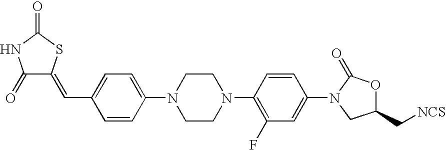

Thiophosgene (0.160 g; 1.39 mmol) was added dropwise to a solution of (S)-N-5-(aminomethyl)-3-[3-fluoro-4-(4-(5-methylene-1,3-thiazolidine-2,4-dione )phenylpiperazin-1-yl)phenyl]-1,3-oxazolidin-2-one (0.385 g, 0.77 mmol) and triethylamine (0.290 g; 2.86 mmol) in dry tetrahydrofuran at 0° C. under nitrogen atmosphere. The reaction mixture warmed to room temperature over 3 hr and then volatiles were removed. The crude product thus obtained was used as such in the Step V.

### STEP V

### Synthesis of (S)-N-3-13-fluoro-4-(4-(5-methylene-1,3-thiazolidine-2,4-dione)phenylpiperazin-1-yl)phenyll-1,3-oxazolidin-2-one-5-methylthio carbamate

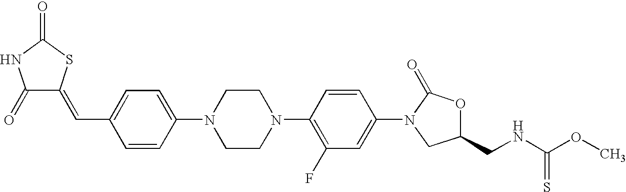

A solution of (S)-N-5-(isothiocyanatomethyl)-3-[3-fluoro-4-(4-(5-methylene-1,3-thiazolidine-2,4-dione)phenylpiperazin-1 -yl)phenyl]-1,3-oxazolidin-2-one (0.4 g, 0.742 mmol) in methanol was refluxed for 24 hr. The reaction mixture was allowed to cool to room temperature and concentrated to gave crude product, which was purified by preparative HPLC to afford the title compound. Yield: 0.057 g; ¹H-NMR (400MHz, DMSO-d₆): 3.1 (s, 3H), 3.4 (s, 4H), 3.7 (m, 2H), 3.8 (s, 3H), 3.9 (s, 1H), 4.1 (m, 1H), 4.8 (m, 1H), 7.12 (m, 3H), 7.2 (m, 2H), 7.4(m, 3H), 7.6 (s, 1H); M/z^{m+1}: 572.

### EXAMPLE 9

### Preparation of (S)-N-3-13-fluoro-4-(4-(5-methylene-1,3-thiazolidine-2,4-dione)phenylpiperazin-1-yl)phenyll-1,3-oxazolidin-2-one-5-methyl carbamate

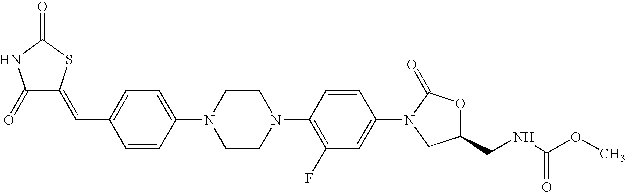

### STEP 1

### Synthesis of (S)-N-5-(aminomethyl)-3-(3-fluoro-4-Bocpiperazin-1-ylphenyl)-1,3-oxazolidin-2-one

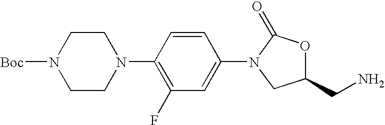

To a solution of (S)-N-5-(azidomethyl)-3-(3-fluoro-4-Bocpiperazin-1-ylphenyl)-1,3-oxazolidin-2-one (2.5 g, 5.95 mmol) in dichloromethane (150 ml) was added 0.190 g of 10% Pd/C and the reaction mixture was hydrogenated at 30 psi for 3 hours. After completion of reaction, the reaction mixture was filtered and concentrated the organic layer to give the title compound. Yield: 2.3 g; M/z^{m+1}: 395.4

### STEP II

### Synthesis of methyl (S)-N-[3-(3-fluoro-4-Bocpiperazin-1-ylphenyl)-2-oxo-1,3-oxazolidin-5-yll methylcarbamate

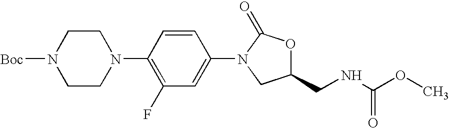

To a solution of (S)-N-5-(aminomethyl)-3-(3-fluoro-4-Bocpiperazin-1-ylphenyl)-1,3-oxazolidin-2-one (1.0 g; 2.5 mmol) in dichloromethane (15 ml), added triethylamine (0.56 g; 5.5 mmol) at 0° C. and stirred for 15 minutes. Methylchloroformate (0.28 g; 3.0 mmol) was added to above reaction mixture at 0° C. and stirred for 30 min at same temperature. After completion of reaction the reaction mixture was diluted with dichloromethane, washed with water, dried over anhydrous sodium sulfate and concentrated to give the title compound. Yield: 0.95 g; M/z^{m+1}: 453.2.

### STEP III

### Synthesis of methyl [(5S)-3-(3-fluoro-4-piperazin-1-ylphenyl)-2-oxo-1,3-oxazolidin-5-yl]methylcarbamate

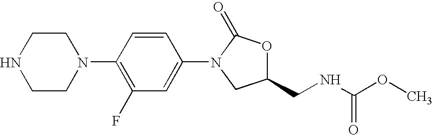

To a solution of methyl (S)-N-[3-(3-fluoro-4-Bocpiperazin-1-ylphenyl)-2-oxo-1,3-oxazolidin-5-yl]methylcarbamate (0.1 g; 0.22 mmol) in dichloromethane (30 ml) dry hydrochloric acid gas was bubbled at 0° C. After completion of reaction, the excess of hydrochloric acid gas was removed by bubbling nitrogen gas. The solvent was removed by distillation to give the title compound. Yield: 0.097g; M/z^{m+1}: 353.2.

### STEP IV

### Synthesis of methyl [(5S)-3-(3-fluoro-4(1-benzaldehyde-4-yl-piperazin-l-ylphenyl)-2-oxo-1,3-oxazolidin-5-yllmethylcarbamate

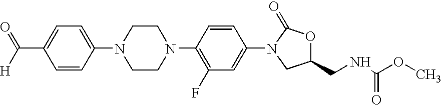

To a solution of methyl [(5S)-3-(3-fluoro-4-piperazin-1-ylphenyl)-2-oxo-1,3-oxazolidin-5-yl]methylcarbamate (0.097 g; 0.25 mmol) in dry dimethylformamide (5 ml), potassium carbonate (0.345 g, 2.5 mmol) was added and stirred for 10 minutes under nitrogen atmosphere at 30° C. To this 4-fluoro benzaldehyde (0.062 g; 0.5 mmol) was added and stirred further at 75° C. for 24 hr. The reaction mixture was quenched with water and extracted with ethyl acetate. Organic layer was washed with water, dried over anhydrous sodium sulfate and concentrated to gave crude product which was purified over silica gel to give the title compound. Yield: 0.045 g; M/z^{m+1}: 457.2.

### STEP V

### Synthesis of (5S)-3-[3-fluoro-4-(4-(5-methylene-1,3-thiazolidine-2,4-dione)phenylpiperazin-1-yl)phenyll-1,3-oxazolidin-2-one-5-methyl carbamate

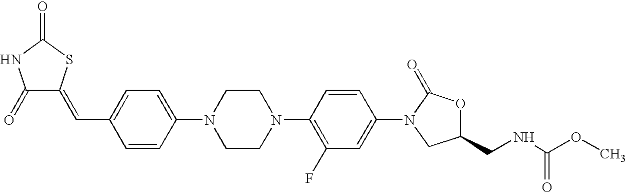

To a solution of methyl [(5S)-3-(3-fluoro-4(1-benzaldehyde-4-yl-piperazin-1-ylphenyl)-2-oxo-1,3-oxazolidin-5-yl]methylcarbamate 0.045 g, 0.098 mmol) in toluene (30 ml) , 2,4-thiazoldinedione (0.016 g, 0.13 mmol), benzoic acid (0.002 g, 0.014 mmol) and piperidine (0.001 g, 0.013 mmol) were added and refluxed the reaction mixture for 1 hr. After completion of reaction, the reaction mixture was allowed to attain 30° C., the solid separated was filtered and dried to give the title compound. Yield: 0.023g; ¹H-NMR (400 MHz, DMSO-d₆): 3.1 (m, 4H), 3.3 (m, 2H), 3.4(m, 4H), 3.5 (m, 3H), 3.7 (m, 1H), 4.0 (m, 1H), 4.7 (m, 1H), 7.12 (m, 4H), 7.4 (m, 3H), 7.6(s, 1H); M/z^{m+1}: 556.2. The following compounds were prepared according to the procedure given in example 9.

| Example No. | Structure | Analytical Data |
|---|---|---|
| 10 | 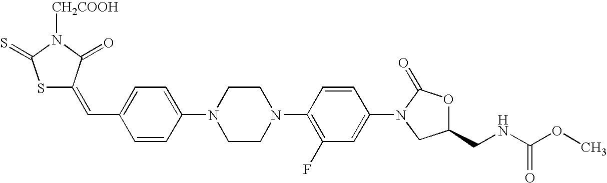 | Yield: 0.120 g; ¹H-NMR (DMSO-d₆): δ 2.9 (m, 2H), 3.1 (m, 4H), 3.5 (m, 7H), 3.7 (m, 1H), 4.0 (m, 1H), 4.3 (m, 1H), 4.7(s, 2H), 7.1 (m, 4H), 7.5 (m, 3H), 7.69 (s, 1H), M/z^{m+1}: 630.2. |
| 11 | 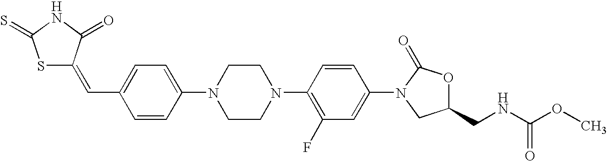 | Yield: 0.073 g; ¹H-NMR (DMSO-d₆): 3.1 (s, 4H), 3.4 (s, 4H), 3.6 (s, 3H), 3.7 (m, 2H), 3.8 (m, 1H), 4.0 (m, 1H), 4.7 (m, 1H), 7.1 (m, 4H), 7.5(m, 4H), M/z^{m+1}: 572.2. |
| 12 | 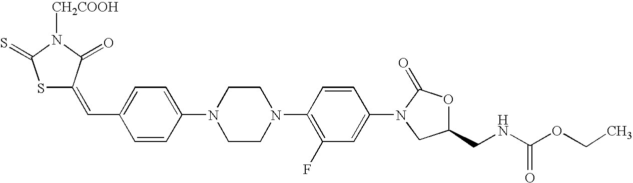 | Yield: 0.587 g; ¹H-NMR (DMSO-d₆): δ 1.1 (t, 3H), 3.1 (m, 4H), 3.3 (m, 2H), 3.5 (m, 4H), 3.71 (m, 1H), 3.98 (m, 2H), 4.0 (m, 1H), 4.56 (s, 2H), 4.7 (m, 1H), 7.1 (m, 4H), 7.4 (m, 3H), 7.7 (s, 1H); M/z^{m+1}: 644.2. |
| 13 | 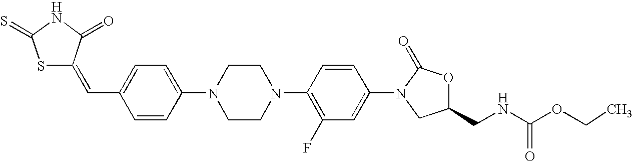 | Yield: 0.455 g; ¹H-NMR (DMSO-d₆): δ 1.1 (t, 3H), 3.1 (s, 4H), 3.3 (m, 2H), 3.5 (s, 4H), 3.8 (m, 1H), 4.0 (m, 2H), 4.1 (m, 1H), 4.7 (m, 1H) 7.1 (m, 4H), 7.4 (m, 4H), M/z^{m+1}: 586.2. |

### EXAMPLE 14

### [(5S)-3-[3-fluoro-4-(4-(5-methy-1,3-thiazolidine-2,4-dione)phenyl piperazin-1-yl)phenyll-1,3-oxazolidin-2-one-5-methyl carbamate

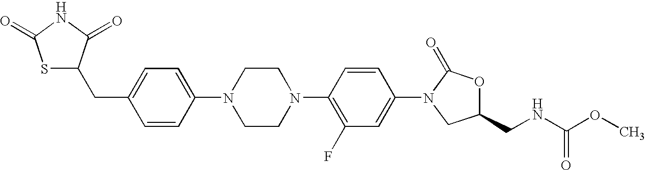

To a solution of (5S)-3-[3-fluoro-4-(4-(5-methylene-1,3-thiazolidine-2,4-dione)phenylpiperazin- 1 -yl)phenyl]-1,3-oxazolidin-2-one-5-methyl carbamate (200 mg, 0.36 mmol) in dichloromethane (300 ml), 10% Pd/C (100 mg) was added and stirred under 20 kg pressure of hydrogen for 24 hr. The reaction mixture was filtered and concentrated to get crude product. The crude product was washed with dichloromethane to furnish the title compound as off white solid. Yield: 0.021 g; ¹H-NMR (DMSO-d₆): 3.0 (m, 1H), 3.05 (m, 4H), 3.2 (m, 5H), 3.5(s, 3H), 3.7(m, 1H), 4.0 (t, 1H), 4.6(m, 1H), 4.8(m, 1H), 6.3(s, 1H), 6.9 (d, 2H), 7.1(m, 3H), 7.19 (dd, 1H), 7.5 (m, 2H); M/z^{m+1}: 557.8. The following compounds were prepared according to the procedure given in example 14.

| Example No. | Structure | Analytical Data |
|---|---|---|
| 15 | 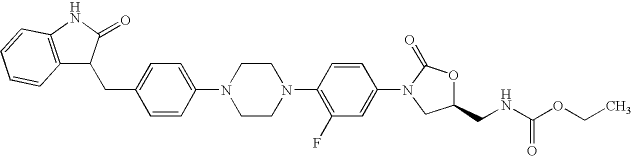 | Yield: 0.070 g; ¹H-NMR (DMSO-d₆): δ 1.23 (t, 3H), 2.9 (m, 1H), 3.21 (m, 4H), 3.32 (m, 3H), 3.40 (m, 1H), 3.7 (m, 5H), 4.03 (t, 1H), 4.12 (q, 2H), 4.78 (m, 1H), 5.12 (m, 1H), 6.85 (m, 6H), 7.09 (m, 4H), 7.47 (d, 1H), 7.54 (s, 1H); M/z^{m+1}: 588. |
| 16 | 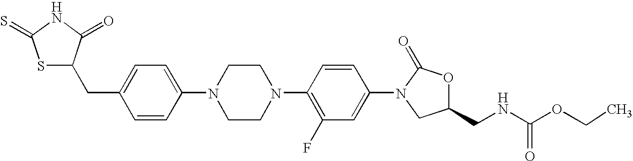 | Yield: 0.100 g; ¹H-NMR (DMSO-d₆): δ 1.1 (t, 3H), 3.1 (m, 5H), 3.2 (m, 5H), 3.3 (d, 2H), 3.7 (m, 1H), 4.0 (m, 3H), 4.7 (m, 1H), 5.0 (m, 1H), 6.9 (m, 2H), 7.1 (m, 2H), 7.18 (m, 1H), 7.4 (m, 2H); M/z^{m+1}: 587.9. |
| 17 | 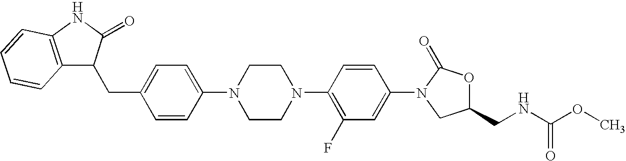 | Yield: 0.058 g; ¹H-NMR (DMSO-d₆): δ 2.17 (s, 3H), 2.89 (m, 1H), 3.2 (m, 4H), 3.31 (m, 3H), 3.52 (m, 3H), 3.68 (m, 2H), 3.78 (m, 1H), 4.03 (t, 1H), 4.76 (m, 1H), 5.17 (m, 1H), 6.85 (m, 6H), 7.09 (m, 4H), 7.47 (d, 1H), 7.54 (s, 1H). M/z^{m+1}: 574 |

### Antimicrobial Testing

The compounds of invention showed in vitro antibacterial activity when tested by the Agar Dilution Method as specified in documents published by the National Committee for Clinical Laboratory Standards (NCCLS), USA.

Briefly, the compounds of invention were weighed, dissolved in Dimethyl Sulfoxide, serially diluted in the same solvent and then incorporated into molten Mueller Hinton Agar in a petridish before solidification, with each petridish containing a different concentration of a compound.

The Bacterial Inoculum was prepared by touching the tops of 3 to 5 well isolated bacterial colonies with the same morphological appearance from an 18 hour old culture with an inoculating loop, transferring the growth to a tube containing 5 ml of normal saline and adjusting the turbidity of the saline suspension to 0.5 Macfarland Turbidity Standard equivalent to a bacterial population of 1.5×10⁸ colony forming units (CFU) per millilitre of suspension.

The bacterial inoculum prepared in the above manner was inoculated onto petri dishes containing Mueller Hinton Agar which had earlier been incorporated with different dilutions of the compounds of invention by a Multipoint Inoculator with each inoculum spot containing approximately 1×10⁴ colony forming units (CFU) of bacteria.

The inoculated petridishes were incubated at 35° C. in an ambient atmosphere for 20 hours. Petridishes containing different concentrations of Vancomycin and Oxacillin and inoculated with Staphylococcus aureus, Coagulase Negative Staphylococci and Enterococci were incubated for 24 hours.

The petridishes after incubation, were placed on a dark non reflecting surface and the Minimum Inhibitory Concentration (MIC) recorded as the concentration which showed no growth of the inoculated culture.

The following minimum inhibitory concentrations (μg/ml) were obtained for representative compounds of the invention which are given in the following table:

### Antimicrobial Screening (MIC) (μg/ml)

| Example | OCID | E. faecium | E. faecalis | S. aureus | S. epidermidis |
|---|---|---|---|---|---|
| No | No | ATCC 700221 | ATCC 29212 | MRO 00001 | MRO 02002 |
| 1 | 0461 | 1 | 2 | 1 | 1 |
| 2 | 0338 | 2 | 2 | 2 | 2 |
| 3 | 0339 | 16 | >16 | >16 | >16 |
| 4 | 0337 | 1 | 1 | 1 | 1 |
| 5 | 0340 | 1 | 2 | 1 | 1 |
| 6 | 0344 | 4 | 4 | 4 | 4 |
| 7 | 0387 | 8 | 8 | 8 | 8 |
| 8 | 0434 | 0.5 | 0.5 | 0.5 | 0.5 |
| 9 | 0363 | 0.5 | 1 | 1 | 2 |
| 10 | 0383 | 8 | 8 | 8 | 8 |
| 11 | 0384 | >16 | >16 | >16 | >16 |
| 12 | 0385 | >16 | >16 | >16 | >16 |
| 13 | 0386 | >16 | >16 | >16 | >16 |
| 14 | 0365 | 4 | 8 | 8 | 8 |
| 15 | 0366 | >16 | >16 | >16 | >16 |
| 16 | 0465 | >16 | >16 | >16 | >16 |
| 17 | 0466 | 4 | 4 | 4 | 4 |

1) E. faecium ATCC 700221-Enterococcus faecium ATCC 700221
2) E. faecalis ATCC 29212-Enterococcus faecalis ATCC 29212
3) S. Epidermis MRO 00001-Staphylococus epidermis Microbial Resource Orchid 00001
4) S. Epidermis MRO 02002-Staphylococus epidermis Microbial Resource Orchid 02002

1. A compound of formula (I) 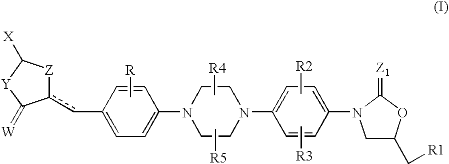
their pharmaceutically acceptable salts, wherein
wherein - - - - represents an optional bond;
W represents O or S;
Y represents NR⁹, S or O, wherein R⁹ represents hydrogen, substituted or unsubstituted alkyl, alkenyl, —CH₂COOR¹⁰, or aryl, or counter ion; wherein R¹⁰represents H or alkyl group;
Z represents CR¹¹ or S;
X represents ═O, ═S or together with R¹¹ forms fused 5 or 6 membered aromatic or heteroaromatic ring system containing carbon atoms or 1 or 2 heteroatoms selected from O, S or N;
Z₁ represents O or S;
R represents substituents selected from cyano, amino, alkyl,alkoxy, nitro, acyl, halogen atom, carboxylic acid or its esters;
R¹ represents halogen, azido, nitro, cyano; AR⁶, where A represents O or S, R⁶ represents hydrogen, substituted or unsubstituted groups selected from alkyl, cycloalkyl, aryl, aralkyl, acyl; N(R^{7a}R^{7b}) where R^{7a} and R^{7b} may be same or different and independently represent hydrogen, formyl, substituted or unsubstituted groups selected from (C₁-C₄)alkyl, aryl, aralkyl, heteroaryl, heteroaralkyl or an aminoacid residue which is attached through acid moiety, or R^{7a} and R^{7b} together with nitrogen may represent a mono or bicyclic saturated or unsaturated ring system which may contain one or more heteroatoms selected from O, S or N; or of the formula —NHC(═B)R⁸ wherein W represents O or S, R⁸ represents hydrogen, substituted or unsubstituted groups selected from (C₁-C₄)alkyl, (C₁-C₄)alkoxy, aryl, (C₃-C₆)cycloalkyl, amino, monoalkylamino, dialkylamino, arylamino, alkylcarbonylamino, arylcarbonylamino, heteroaryl, heterocyclyl, heteroaralkyl, or R¹ is of the formula —NHS(O)ₚ(C₁-C4)alkyl, —NHS(O)paryl or —NHS(O)ₚheteroaryl, where p is 0 to 2;
R² and R³ may be same or different and independently represent hydrogen, halogen, hydroxy, alkyl or alkoxy;
R⁴ and R⁵ may be same or different and independently represent hydrogen, cyano, nitro, amino, halogen, hydroxy, substituted or unsubstituted groups selected from (C₁-C₄)alkyl, haloalkyl, (C₁-C₄)alkoxy, (C₁-C₄)alkylthio, (C₃-C₆)cycloalkyl or either of R⁴ or R⁵ represent an oxo or thiooxo group.

2. A compound of formula (I) as claimed in claim 1, which is selected from:
a) (S)-N-[3-[3-fluoro-4-[4-[4-[(3-carboxymethyl-5-oxo-2-thioxo-1,3-thiazolidin-3-yl)methine]phenyl]piperazin-1 -yl]phenyl]-2-oxooxazolidin-5-ylmethyl] acetamide;
b) (S)-N-[3-[3-fluoro-4-[4-[4-[(1,3-dihydro-2-oxoindol-3-yl)methine]phenyl] piperazin-1-yl]phenyl]-2-oxooxazolidin-5-ylmethyl] acetamide;
c) (S)-N-[3-[3-fluoro-4-[4-[4-[(2-thioxo-4-oxo-1,3-thiazolidin-4-yl)methine] phenyl]piperazin-1 -yl]phenyl]-2-oxooxazolidin-5-ylmethyl] acetamide;
d) (S)-N-[3-[3-fluoro-4-[4-[4-[(2,4-dioxo-1,3-thiazolidin-4-yl)methine]phenyl] piperazin-1-yl]phenyl]-2-oxooxazolidin-5-ylmethyl] acetamide;
e) (S)-N-[3-[3-fluoro-4-[4-[4-[(1,3-dihydro-2-oxoindol-3-yl)methylene]phenyl] piperazin-1-yl]phenyl]-2-oxooxazolidin-5-ylmethyl] acetamide;
f) (S)-N-[3-[3-fluoro-4-[4-[4-[(2-thioxo-4-oxo-1,3-thiazolidin-4-yl)methylene] phenyl]piperazin-1-yl]phenyl]-2-oxooxazolidin-5-ylmethyl]acetamide;
g) (S)-N-[3-[3-fluoro-4-(4-(5-methylene-1,3-thiazolidine-2,4-dione)phenyl piperazin -1-yl)phenyl]-1,3-oxazolidin-2-one-5- methylthiocarbamate;
h) (S)-N-[3-[3-fluoro-4-(4-(5-methylene-1,3-thiazolidine-2,4-dione )phenyl piperazin-1 -yl)phenyl]-1,3-oxazolidin-2-one-5- methylcarbamate;
i) (S)-N-[3-[3-fluoro-4-[4-[4-[(3-carboxymethyl-5-oxo-2-thioxo-1,3-thiazolidin-3-yl)methine]phenyl]piperazin-1 -yl]phenyl]-2-oxooxazolidin-5-ylmethyl] carbamate;
j) (S)-N-[3-[3-fluoro-4-[4-[4-[(5-oxo-2-thioxo-1,3-thiazolidin-3-yl)methine] phenyl]piperazin-1-yl]phenyl]-2-oxooxazolidin-5-ylmethyl] carbamate;
k) (S)-N-[3-[3-fluoro-4-[4-[4-[(3-carboxymethyl-5-oxo-2-thioxo-1,3-thiazolidin-3-yl)methine]phenyl]piperazin-1 -yl]phenyl]-2-oxooxazolidin-5-yl-ethyl] carbamate;
1) (S)-N-[3-[3-fluoro-4-[4-[4-[(5-oxo-2-thioxo-1,3-thiazolidin-3-yl) methine]phenyl]piperazin-1 -yl]phenyl]-2-oxooxazolidin-5-yl-ethyl] carbamate;
m) (S)-N-[3-[3-fluoro-4-[4-[4-[(1,3-dihydro-2-oxoindol-3-yl)methylene]phenyl] piperazin-1-yl]phenyl]-2-oxooxazolidin-5-ylmethyl] carbamate;
n) (S)-N-[3-[3-fluoro-4-[4-[4-[(1,3-dihydro-2-oxoindol-3-yl)methylene]phenyl] piperazin-1-yl]phenyl]-2-oxooxazolidin-5-yl-ethyl] carbamate;
o) (S)-N-[3-[3-fluoro-4-[4-[4-[(2-thioxo-4-oxo-1,3-thiazolidin-4-yl)methine] phenyl]piperazin-1-yl]phenyl]-2-oxooxazolidin-5-yl-ethyl] carbamate;
p) (S)-N-[3-[3-fluoro-4-[4-[4-[(2-thioxo-4-oxo-1,3-thiazolidin-4-yl)methine] phenyl]piperazin-1-yl]phenyl]-2-oxooxazolidin-5-ylmethyl] carbamate.

3. A method of treating or preventing an infectious disorder in a human or animal, comprising administering an effective amount of a compound of claim 1 to human or animal in need thereof.

4. A method of treating or preventing an infectious disorder in a human or animal, comprising administering an effective amount of a compound of claim 2 to human or animal in need thereof

5. A method as claimed in claim 4, wherein the infectious disorder is caused by bacteria.
